(19) United States
(12) Patent Application Publication
(10) Pub. No.: US 2025/0017970 A1
(43) Pub. Date: Jan. 16, 2025

(54) Reduced Expression of Sarm1 For Use In Cell Therapy

(71) Applicant: EMENDOBIO INC., Wilmington, DE (US)

(72) Inventors: Rafi EMMANUEL, Ramla (IL); Michal GOLAN MASHIACH, Ness-Ziona (IL); Rachel DIAMANT, Ein Vered (IL)

(21) Appl. No.: 18/715,348

(22) PCT Filed: Dec. 1, 2022
(86) PCT No.: PCT/US2022/080749

§ 371 Date: May 31, 2024

Related U.S. Application Data
(60) Provisional application No. 63/284,995, filed on Dec. 1, 2021.

(51) Int. Cl. A61K 35/17; C12N 5/0783; C12N 9/22; C12N 15/11

(52) U.S. Cl. CPC A61K 35/17; C12N 5/0646; C12N 9/22; C12N 15/111; C12N 2310/20

(57) ABSTRACT

The invention relates to a method for adoptive cell therapy or prophylaxis comprising administering SARM1-inhibited or SARM1-inactivated cells to a subject suffering from or determined to be at risk of suffering from a cancer, infection, disease, or disorder.

This application claims the benefit of U.S. Provisional Application No. 63/284,995, filed Dec. 1, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “221201_91818-A-PCT_Sequence_Listing_AWG.xml”, which is 11,660 kilobytes in size, and which was created on Dec. 1, 2022 in the IBM-PC machine format, having an operating system compatibility with MS-Windows, which is contained in the XML file filed Dec. 1, 2022 as part of this application.

### BACKGROUND OF INVENTION

Immunotherapies such as CAR T-cell therapies provide a promising approach for treating several diseases and disorders. However, to make such therapies more effective, it is highly desirable to develop an allogeneic adoptive transfer strategy in which the engineered cells have increased persistence upon transfer to the patient.

### SUMMARY OF THE INVENTION

The sterile alpha and TIR motif-containing 1 (SARM1) gene is a NAD+ hydrolase that acts as a negative regulator of MYD88- and TRIF-dependent toll-like receptor signaling pathway by promoting Wallerian degeneration, an injury-induced form of programmed subcellular death. SARM1 can also activate cell death in response to stress (Molday et al., 2013).

Disclosed are approaches for knocking out, knocking down, or inhibiting expression of the SARM1 gene in engineered immune cell types to increase their persistence, proliferation, and/or retention during cell therapy. Accordingly, biallelic knockout, knockdowns, or inhibition of expression of the SARM1 gene in cell therapy-related cell types as described herein may be utilized to improve cell therapy approaches, including for the treatment of cancers.

The present disclosure also provides a method for adoptive cell therapy or prophylaxis comprising administering SARM1-inhibited or SARM1-inactivated cells to a subject suffering from or determined to be at risk of suffering from a cancer, infection, disease, or disorder. In some embodiments, the cells are immune cells (e.g., lymphocyte, monocyte, or macrophages). In some embodiments the SARM1-inhibited immune cells are autologous. In other embodiments the SARM1-inhibited immune cells are allogeneic.

### BRIEF DESCRIPTION OF THE DRAWINGS

FIGS. 1A-1B: Screen for activity of RNA guide molecules targeting SARM1 in HeLa cells. Cells were harvested 72 h post DNA transfection. Genomic DNA was extracted and used for capillary electrophoreses after amplifying the endogenous genomic regions using on-target primers. The graph represents the average of % editing±standard deviation (STDV) of three (3) independent experiments. FIG. 1A: An SpCas9 coding plasmid was co-transfected with a plasmid expressing the indicated RNA guide molecule. FIG. 1B: An OMNI-50 or OMNI-79 CRISPR nuclease was co-transfected with the indicated RNA guide molecule.

FIG. 2: RNPs of a SpCas9 nuclease complexed with a specific RNA guide molecule were electroporated into Neuro-2a cells to determine RNP activity. Cells were harvested 72 hours post DNA electroporation, genomic DNA was extracted, and then analyzed by next-generation sequencing (NGS). The graph editing represents the % of editing±STDV of two (2) independent electroporations.

FIG. 3: Relative amount of SARM1 RNA after editing. Neuro-2a cells were harvested seven (7) days post electroporation, RNA was extracted and reverse transcribed. The relative amount of SARM1 RNA was quantified using AriaMx system. The level of the mRNA is quantified relative to untreated cells that were not edited.

FIG. 4: Editing activity of OMNI-103 CRISPR nuclease with an RNA guide molecule targeting SARM1 in HeLa cells. Specific RNA guide molecules were-co-transfected with OMNI-103 CRISPR nuclease to determine their on-target activity. Cells were harvested 72 hours post DNA transfection, genomic DNA was extracted, and the region of the mutation was amplified and analyzed by NGS. Transfection efficiency was measured by mCherry fluorescence. The graph represents the % of editing±STDV of three (3) independent transfections.

FIG. 5: SARM1 pathway diagram.

FIG. 6A: SARM1 editing and NGS results. FIG. 6B: Killing assay. FIG. 6C: Viability (ATPlite) assay.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target

DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-13457. Each possibility represents a separate embodiment. In some of these embodiments, the guide sequence portion comprises a sequence that is the same as a sequence set forth in any of SEQ ID NOs: 1-13457. Throughout this application, the terms “guide molecule,” “RNA guide molecule.” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common to all alleles of a gene.

In embodiments of the present invention, an RNA molecule targeting SARM1 comprises a guide sequence portion having 17-50 contiguous nucleotides containing nucleotides in the sequence set forth in any one of SEQ ID NOs: 1-13457. The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, a guide sequence portion of an RNA molecule targeting SARM1 may be 50 nucleotides in length and contain 20-22 contiguous nucleotides in the sequence set forth in any one of SEQ ID NOs: 1-13457. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-13457. For example, a guide sequence portion having 17 nucleotides in the sequence of 17 contiguous nucleotides set forth in SEQ ID NO: 13458 may consist of any one of the following nucleotide sequences (nucleotides excluded from the contiguous sequence are marked in strike-through):

(SEQ ID NO: 13458)
AAAAAAAUGUACUUGGUUCC
17 nucleotide guide sequence 1:
(SEQ ID NO: 13459) [symbol]
AAAAUGUACUUGGUUCC
17 nucleotide guide sequence 2:
(SEQ ID NO: 13460) [symbol]
AAAAAUGUACUUGGUUC [symbol]
17 nucleotide guide sequence 3:
(SEQ ID NO: 13461) [symbol]
AAAAAAUGUACUUGGUU [symbol]
17 nucleotide guide sequence 4:
(SEQ ID NO: 13462)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24 or 25 nucleotides in length. In such embodiments the guide sequence portion comprises 17-50 nucleotides containing the sequence of 20, 21 or 22 contiguous nucleotides set forth in any one of SEQ ID NOs: 1-13457 and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single-guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

Any one of, or combination of, any strategy for reducing or deactivating SARM1 expression in cells for use in cell therapy may be used in the context of the invention.

According to embodiments of the present invention, there is provided a method for adoptive cell therapy or prophylaxis comprising administering SARM1-inhibited or SARM1-inactivated cells to a subject suffering from or determined to be at risk of suffering from a cancer, infection, disease, or disorder.

In some embodiments, the SARM1-inhibited or SARM1-inactivated cells are modified to have reduced or inactivated expression of SARM1, or the cells are modified to express a dominant negative SARM1 sequence variant or dominant negative fragment thereof.

In some embodiments, the cells are selected from the group consisting of hematopoietic stem cells (HSCs), induced pluripotent stem cells (iPS cells), iPSc-derived cells, natural killer cells (NKs), iPS-derived NK cells (iNKs), T cells, innate-like T cells (iTs), natural killer T cells (NKTs), γδ T cells, iPSc-derived T cells, invariant NKT cells (iNKTs), iPSc-derived NKTs, monocytes, or macrophages. In some embodiments, progenitor cells are first modified and then differentiated to create the SARM1-inhibited or SARM1-inactivated cells. Alternatively, the SARM1-inhibited or SARM1-inactivated cells may be created by directly editing cells, e.g. primary NK cells.

In some embodiments, the SARM1-inhibited or SARM1-inactivated cells display increased functionality relative to counterpart cells that have wild-type or unmodified SARM1 expression. In some embodiments, the SARM1-inhibited or SARM1-inactivated cells display increased and/or enhanced viability relative to counterpart cells that have wild-type or unmodified SARM1 expression. In some embodiments, the SARM1-inhibited or SARM1-inactivated cells display enhanced functionality in vivo, such as killing ability, cytotoxicity, trafficking, localization, persistence, and/or proliferation relative to counterpart cells that have wild-type or unmodified SARM1 expression.

In some embodiments, the SARM1-inhibited or SARM1-inactivated cells display increased functionality, increased viability, increased persistence, increased proliferation, and/or increased tumor retention in the subject relative to counterpart cells that have wild-type or unmodified SARM1 expression.

In some embodiments, the SARM1-inhibited or SARM1-inactivated cells display increased cytotoxic activity and/or increased killing activity in the subject relative to counterpart cells that have wild-type or unmodified SARM1 expression. For example, in some embodiments a SARM1-inhibited or SARM1-inactivated cell displays higher killing activity of a target cancel relative to a counterpart cell that has wild-type or unmodified SARM1 expression.

In some embodiments, the subject is suffering from or determined to be at risk of suffering from a cancer.

In some embodiments, the cancer comprises a tumor and/or the cancer is a hematological malignancy.

In some embodiments, the cancer is selected from the group consisting of static melanoma, metastatic prostate cancer, metastatic breast cancer, triple negative breast cancer, bladder cancer, brain cancer, esophageal cancer, liver cancer, head and neck cancer, squamous cell lung cancer, non-small lung cell cancer, Merkel cell carcinoma, sarcoma, hepatocellular cancer, multiple myeloma, leukemia, non-Hodgkin's lymphoma, lymphoma, B cell lymphoma, acute myeloid leukemia, pancreatic cancer, colorectal carcinoma, cervical cancer, gastric carcinoma, kidney cancer, metastatic renal cell carcinoma, leukemia, ovarian cancer, and malignant glioma.

In some embodiments, the SARM1-inhibited or SARM1-inactivated cells are created ex vivo or in vitro.

some embodiments, the SARM1-inhibited or SARM1-inactivated cells are created in vivo.

In some embodiments, the SARM1-inhibited or SARM1-inactivated cells are created from cells obtained from the subject by mobilization and/or by apheresis.

In some embodiments, the cells are obtained the cells from the subject by bone marrow aspiration.

In some embodiments, the cells are prestimulated prior to the SARM1 inhibition or SARM1 inactivation of the cells.

In some embodiments, the SARM1-inhibited or SARM1-inactivated cells are culture expanded prior to administration to the subject.

In some embodiments, the SARM1-inhibited or SARM1-inactivated cells are capable of engraftment.

In some embodiments, the SARM1-inhibited or SARM1-inactivated cells are capable of giving rise to progeny cells.

In some embodiments, the SARM1-inhibited or SARM1-inactivated cells are capable of giving rise to progeny cells after an engraftment.

In some embodiments, the SARM1-inhibited or SARM1-inactivated cells are capable of giving rise to progeny cells after an autologous engraftment.

In some embodiments, the SARM1-inhibited or SARM1-inactivated cells are capable of giving rise to progeny cells for at least 12 months or at least 24 months after engraftment.

In some embodiments, the SARM1-inhibited or SARM1-inactivated cells are created by delivering a gapmer, shRNA, siRNA, a customized TALEN, meganuclease, zinc finger nuclease, CRISPR nuclease, or a small molecule inhibitor to cells.

In some embodiments, alleles of the SARM1 gene in the SARM1-inhibited or SARM1-inactivated cells are subjected to an insertion or deletion mutation.

In some embodiments, the insertion or deletion mutation creates an early stop codon.

In some embodiments, SARM1-inhibited or SARM1-inactivated cells are created by a method comprising
introducing to the cells a composition comprising:
   at least one CRISPR nuclease, or a nucleotide molecule encoding a CRISPR nuclease; and
   an RNA molecule comprising a guide sequence portion, or a nucleotide molecule encoding the RNA molecule,
wherein a complex of the CRISPR nuclease and the RNA molecule affects a double strand break in alleles of the SARM1 gene,
wherein the guide sequence portion of the RNA molecule comprises 17-50 contiguous nucleotides.

In some embodiments, guide sequence portion is complementary to a target sequence located from 50 base pairs upstream to 50 base pairs downstream of Exon I, Exon II, Exon III, Exon IV, Exon V, Exon VI, Exon VII, Exon VIII, or Exon IX of the SARM1 gene.

In some embodiments, the guide sequence portion is complementary to a target sequence located from 30 base pairs upstream to 30 base pairs downstream of an Exon of the SARM1 gene, and
a) the Exon is Exon I and the guide sequence portion comprises a sequence that is the same as or differs by no more than 3 nucleotides from a sequence set forth in any of SEQ ID NOs: 1-56, 301-356, 1348-2223, 57-114, 357-414, 2224-3095, 115-174,415-474, 3096-3963;
b) the Exon is Exon II, and the guide sequence portion comprises a sequence that is the same as or differs by no more than 3 nucleotides from a sequence set forth in any of SEQ ID NOs: 3964-7683;
c) the Exon is Exon III, and the guide sequence portion comprises a sequence that is the same as or differs by no more than 3 nucleotides from a sequence set forth in any of SEQ ID NOs: 7684-8967;
d) the Exon is Exon IV, and the guide sequence portion comprises a sequence that is the same as or differs by no more than 3 nucleotides from a sequence set forth in any of SEQ ID NOs: 8968-9525;
e) the Exon is Exon V, and the guide sequence portion comprises a sequence that is the same as or differs by no more than 3 nucleotides from a sequence set forth in any of SEQ ID NOs: 9526-10947;
f) the Exon is Exon VI, and the guide sequence portion comprises a sequence that is the same as or differs by no more than 3 nucleotides from a sequence set forth in any of SEQ ID NOs: 10948-11571;
g) the Exon is Exon VII, and the guide sequence portion comprises a sequence that is the same as or differs by no more than 3 nucleotides from a sequence set forth in any of SEQ ID NOs: 11572-12717;
h) the Exon is Exon VIII, and the guide sequence portion comprises a sequence that is the same as or differs by no more than 3 nucleotides from a sequence set forth in any of SEQ ID NOs: 12718-13455; or
i) the Exon is Exon IX, and the guide sequence portion comprises a sequence that is the same as or differs by no more than 3 nucleotides from a sequence set forth in any of SEQ ID NOs: 598-1347.

In some embodiments, the guide sequence portion comprises 17-50 contiguous nucleotides containing nucleotides in the sequence set forth in any one of SEQ ID NOs: 1-13457.

In some embodiments, the SARM1-inhibited or SARM1-inactivated cells exhibit increased viability and/or increased functionality compared to counterpart cells that have wild-type or unmodified SARM1 expression.

According to embodiments of the present invention, there is provided a medicament comprising SARM1-inhibited or SARM1-inactivated cells for use in treating or preventing a cancer, infection, disease, or disorder in a subject according to the methods presented herein. In some embodiments, the present invention provides a kit for treating or preventing a cancer, infection, disease, or disorder in a subject, comprising the medicament and instructions for delivering the composition to a subject suffering from or determined to be at risk of suffering from a cancer, infection, disease, or disorder.

According to embodiments of the present invention, there is provided a method for inactivating alleles of the sterile alpha and toll/interleukin-1 receptor motif-containing 1 (SARM1) gene in a cell, the method comprising
introducing to the cell a composition comprising:
   at least one CRISPR nuclease, or a nucleotide molecule encoding a CRISPR nuclease; and
   an RNA molecule comprising a guide sequence portion, or a nucleotide molecule encoding the RNA molecule,
wherein a complex of the CRISPR nuclease and the RNA molecule affects a double strand break in alleles of the SARM1 gene,
wherein the guide sequence portion comprises 17-50 contiguous nucleotides containing nucleotides in the sequence set forth in any one of SEQ ID NOs: 1-13457, and
wherein the cell is selected from the group consisting of a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPSc-derived T cell, invariant NKT cell (iNKT), iPSc-derived NKT, monocyte, or macrophage.

According to embodiments of the present invention, there is provided a cell modified by the method for inactivating alleles of the SARM1 gene in a cell, wherein the modified cell comprises at least one inactivated SARM1 allele.

In some embodiments, the modified cell comprises two inactivated SARM1 alleles. In some embodiments, the modified cell exhibits increased viability and/or increased functionality compared to a counterpart cell that has wild-type or unmodified SARM1 expression. In some embodiments, increased functionality comprises increased cytotoxicity and/or increased killing capability.

In some embodiments, the cell is for use in adoptive cell therapy or prophylaxis.

In some embodiments, the adoptive cell therapy or prophylaxis is to treat or prevent a cancer, infection, disease, or disorder in a subject.

According to embodiments of the present invention, there is provided a method of inactivating SARM1 expression in a cell by delivering an RNA molecule comprising a guide sequence portion (e.g. a targeting sequence) comprising a nucleotide sequence that is fully or partially complementary to a target sequence comprising a SNP position (REF/SNP sequence) located in or near an allele of the SARM1 gene. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a target sequence and provide a cleavage event, by a CRISPR nuclease complexed therewith, selected from a double-strand break and a single-strand break within 500, 400, 300, 200, 100, 50, 25, or 10 nucleotides of a SARM1 target site. In some embodiments, the cleavage event enables non-sense mediated decay of the SARM1 gene. In some embodiments, the RNA molecule is a guide RNA molecule such as a crRNA molecule or a single-guide RNA molecule.

In some embodiments, the target sequence of an allele of SARM1 gene is altered (e.g., by introduction of an NHEJ-mediated indel (e.g., insertion or deletion), and results in reduction or elimination of expression of the gene product encoded by the allele of SARM1 gene. In some embodiments, the reduction or elimination of expression is due to non-sense mediated mRNA decay such as due to immature stop codon. In some embodiments, the reduction or elimination of expression is due to expression of a truncated form of the SARM1 gene product. In some embodiments, the guide sequence portion is complementary to a target sequence comprising a SNP position. In some embodiments, the SNP position is rs782593684. In some embodiments, the guide sequence portion comprises a sequence that is the same as or differs by no more than 1, 2, or 3 nucleotides from a sequence set forth in any of SEQ ID NOs: 1-174. In some embodiments, the guide sequence portion comprises a sequence that is the same as or differs by no more than 1, 2, or 3 nucleotides from a sequence set forth in any of SEQ ID NOs: 1, 4, 8, 20, 24, 26-27, 33, 35, 37, 41, 47, 49, 51, 53, 56, 175-214, 57, 60, 64, 76, 80, 82-83, 92, 94, 98, 102, 105, 107, 109, 111, 114, 215-256, 115, 118, 122, 132, 139, 141-142, 151, 157, 161, 164-165, 167, 169, 171, 174, and 257-300. Each possibility represents a separate embodiment. In some embodiments, the SNP position 17:28372349_C_CT. In some embodiments, the guide sequence portion comprises a sequence that is the same as or differs by no more than 1, 2, or 3 nucleotides from a sequence set forth in any of SEQ ID NOs: 301-474. In some embodiments, the guide sequence portion comprises a sequence that is the same as or differs by no more than 1, 2, or 3 nucleotides from a sequence set forth in any of SEQ ID NOs: 301, 304, 315, 321, 326-328, 335, 338, 340, 344, 346-348, 353, 355-356, 475-513, 357, 361, 364, 368, 373, 384-385, 393, 396, 398, 402, 404-406, 411, 413-414, 514-554, 415-416, 419, 422, 424, 427, 432, 443-444, 458, 462, 464-466, 471, 473-474, and 555-597. Each possibility represents a separate embodiment.

According to embodiments of the present invention, there is provided an RNA molecule, or a sequence encoding the RNA molecule, wherein the RNA molecule comprises a guide sequence portion (e.g. a targeting sequence) comprising a nucleotide sequence that is fully or partially complementary to a target sequence located in or near the SARM1 gene. In some embodiments, the guide sequence portion is complementary to a target sequence located from 30 base pairs upstream to 30 base pairs downstream of Exon I, Exon II, Exon III, Exon IV, Exon V, Exon VI, Exon VII, Exon VIII, or Exon IX of the SARM1 gene. In some embodiments, the guide sequence portion is complementary to a target sequence located from 50 base pairs upstream to 50 base pairs downstream of Exon I, Exon II, Exon III, Exon IV, Exon V, Exon VI, Exon VII, Exon VIII, or Exon IX of the SARM1 gene. Each possibility represents a separate embodiment. In some embodiments, the target sequence of SARM1 gene is altered (e.g., by introduction of an NHEJ-mediated indel (e.g., insertion or deletion), and results in reduction or elimination of expression of the gene product encoded by the SARM1 gene. In some embodiments, the reduction or elimination of expression is due to non-sense mediated mRNA decay. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a target sequence and provide a cleavage event, by a CRISPR nuclease complexed therewith, selected from a double-strand break and a single-strand break within 500, 400, 300, 200, 100, 50, 25, or 10 nucleotides of a SARM1 target site. In some embodiments, the cleavage event enables non-sense mediated decay of the SARM1 gene. In some embodiments, the RNA molecule is a guide RNA molecule such as a crRNA molecule or a single-guide RNA molecule.

In some embodiments, the guide sequence portion is complementary to a target sequence located from 30 base pairs upstream to 30 base pairs downstream of an Exon of the SARM1 gene. In some embodiments, the guide sequence portion is complementary to a target sequence located from 50 base pairs upstream to 50 base pairs downstream of an Exon of the SARM1 gene. In some embodiments, the guide sequence portion is complementary to a target sequence located from 7 base pairs upstream to 7 base pairs downstream of an Exon of the SARM1 gene. In some embodiments, the Exon is Exon I and the guide sequence portion comprises a sequence that is the same as or differs by no more than 3 nucleotides from a sequence set forth in any of SEQ ID NOs: 1-56, 301-356, 1348-2223, 57-114, 357-414, 2224-3095, 115-174, 415-474, and 3096-3963. In some embodiments, the Exon is Exon II, and the guide sequence portion comprises a sequence that is the same as or differs by no more than 3 nucleotides from a sequence set forth in any of SEQ ID NOs: 3964-7683. In some embodiments, the Exon is Exon III, and the guide sequence portion comprises a sequence that is the same as or differs by no more than 3 nucleotides from a sequence set forth in any of SEQ ID NOs: 7684-8967. In some embodiments, the Exon is Exon IV, and the guide sequence portion comprises a sequence that is the same as or differs by no more than 3 nucleotides from a sequence set forth in any of SEQ ID NOs: 8968-9525. In some embodiments, the Exon is Exon V, and the guide sequence portion comprises a sequence that is the same as or differs by no more than 3 nucleotides from a sequence set forth in any of SEQ ID NOs: 9526-10947. In some embodiments, the Exon is Exon VI, and the guide sequence portion comprises a sequence that is the same as or differs by no more than 3 nucleotides from a sequence set forth in any of SEQ ID NOs: 10948-11571. In some embodiments, the Exon is Exon VII, and the guide sequence portion 30) comprises a sequence that is the same as or differs by no more than 3 nucleotides from a sequence set forth in any of SEQ ID NOs: 11572-12717. In some embodiments, the Exon is Exon VIII, and the guide sequence portion comprises a sequence that is the same as or differs by no more than 3 nucleotides from a sequence set forth in any of SEQ ID NOs: 12718-13455. In some embodiments, the Exon is Exon IX, and the guide sequence portion comprises a sequence that is the same as or differs by no more than 3 nucleotides from a sequence set forth in any of SEQ ID NOs: 598-1347.

In embodiments of the present invention, an RNA molecule is used to direct a CRISPR nuclease to an exon or a splice site of a SARM1 allele in order to create a double-stranded break (DSB), leading to insertion or deletion of nucleotides by inducing an error-prone non-homologous end-joining (NHEJ) mechanism and formation of a frameshift mutation in the SARM1 allele. The frameshift mutation may result in, for example, inactivation or knockout of the SARM1 allele by generation of an early stop codon in the SARM1 allele and to generation of a truncated protein or to nonsense-mediated mRNA decay of the transcript of the allele. In further embodiments, one RNA molecule is used to direct a CRISPR nuclease to a promotor of a SARM1 allele. CRISPR compositions used to inactivate SARM1 alleles as described herein may include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

According to some aspects, there is provided a SARM1-inhibited or a SARM1-inactivated cell. In some embodiments, the cell is an immune cell (e.g., monocyte, macrophage, lymphocyte, natural killer cell (NK), iPS-derived NK cell (iNK), T cell, innate-like T cell (iT), iPS-derived T cell, natural killer T cell (NKT), invariant NKT cell (iNKT), or iPS-derived NKT cell). In some embodiments, the SARM1-inhibited cell is a stem cell, HSC, or iPSc. Each possibility represents a separate embodiment. The SARM1-inhibited or SARM1-inactivated cell (e.g., immune cells) may be generated by utilizing any suitable SARM1 inhibitor including a peptide, a polypeptide, a protein, a nuclease, a small molecule, or a polynucleotide.

In some embodiments, the SARM1-inhibited or SARM1-inactivated cell is genetically modified to have reduced activity of SARM1. In some embodiments, the SARM1-inhibited or SARM1-inactivated cell is derived from a precursor cell that was genetically modified to have reduced activity of SARM1. In a non-limiting example, SARM1-inhibited iNK is derived from iPSc that was genetically modified to have reduced activity of SARM1 such as by utilizing CRISPR to knockout SARM1 alleles and further differentiated into an iNK cell.

In some embodiments, the SARM1-inhibited or SARM1-inactivated cells, or cells derived therefrom are used for cell therapy. In some embodiments, the SARM1-inhibited cells, or cells derived therefrom are used for immunotherapy.

According to some aspects of the present disclosure, provided herein is a method for increasing cell viability and/or cell functionality by inactivating alleles of the sterile alpha and toll/interleukin-1 receptor motif-containing 1 (SARM1) gene in a cell, the method comprising introducing to the cell a composition comprising: a CRISPR nuclease, or a nucleotide molecule encoding a CRISPR nuclease, and an RNA molecule comprising a guide sequence portion 17-50 contiguous nucleotides containing nucleotides in the sequence set forth in any one of SEQ ID NOS: 1-13457, or a nucleotide molecule encoding the RNA molecule, wherein a complex of the CRISPR nuclease and the RNA molecule affects a double strand break in alleles of the SARM1 gene. Non-limiting examples of cell types which may be modified by this method include, but are not limited to, liver cells (e.g., hepatocytes), lung cells, spleen cells, pancreas cells, colon cells, skin cells, bladder cells, eye cells, ocular cells, retinal cells, corneal cells, brain cells, esophagus cells, cells of the head, cells of the neck, cells of the ovary, cells of the testes, prostate cells, placenta cells, epithelial cells, endothelial cells, adipocyte cells, kidney/renal cells, heart cells, muscle cells, blood cells (e.g., white blood cells), immune cells, central nervous system (CNS) cells, ganglion cells, and the like, as well as combinations of the foregoing.

### SARM1 Editing Strategies

The provided methods to knockout SARM1 alleles in a cell may be used to create SARM1 inactivated cells for use in cell therapy or immunotherapy.

SARM1 editing strategies include, but are not limited to: (1) Biallelic knockout by targeting any one of, or a combination of, Exons 2-9, including within seven nucleotides upstream and downstream of the exons to flank splice donor and acceptor sites, as frameshifts in these exons lead to non-functional, truncated SARM1 proteins or non-sense mediated decay of the mutated SARM1 transcripts; and (2) Truncation of SARM1 protein by mediating indels in Exon 1 upstream to or overlapping the second methionine codon in the exon that would eliminate it and thus prevent re-initiation of translation, or by disrupting a splice donor by targeting Exon 1-Intron 1 junction. CRISPR Nucleases and PAM Recognition

In some embodiments, the sequence specific nuclease is selected from CRISPR nucleases, or is a functional variant thereof. In some embodiments, the sequence specific nuclease is an RNA-guided DNA nuclease. In such embodiments. the RNA sequence which guides the RNA-guided DNA nuclease (e.g., Cpf1) binds to and/or directs the RNA-guided DNA nuclease to all SARM1 alleles in a cell. In some embodiments, the CRISPR complex does not further comprise a tracrRNA. In a non-limiting example, in which the RNA-guided DNA nuclease is a CRISPR protein, the at least one nucleotide which differs between the dominant SARM1 allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition. there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Publication No. 2015/0211023, the entire content of which is incorporated by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Case (or CasD), Cas6, Cas6e, Cas6f, Cas7, Cas8a1, Cas8a2,Cas8b, Cas8c, Cas9, Cas10, Cas1 Od, CasF, CasG, CasH, Csy1, Csy2, Csy3, Cse1 (or CasA), Cse2(or CasB), Cse3 (or CasE), Cse4 (or CasC), Csc1, Csc2, Csa5, Csn2, Csm2, Csm3, Csm4, Csm5,Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA-guided DNA nuclease of a CRISPR system, such as a Cas9 protein or modified Cas9 or homolog or ortholog of Cas9, or other RNA-guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, the content of each of which are incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA-guided DNA nuclease of a Type II CRISPR System, such as a Cas9 protein or modified Cas9 or homologs, orthologues, or variants of Cas9, or other RNA-guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA-guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2″-O-methylcytidine, 5-carboxy methylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxy carbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2-O)-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

In addition to targeting SARM1 alleles by a RNA-guided CRISPR nuclease, other means of inhibiting SARM1 expression in a target cell for use in cell therapy (e.g. a hematopoietic stem cell (HSC), induced pluripotent stem cells (iPS cells), natural killer cell (NK), iPSc-derived NK cell (iNK), T cell, innate-like T cell (iT), γδ T cells, iPSc-derived T cell, natural killer T cell (NKT), invariant NKT cell (iNKT), iPSc-derived NKT, monocyte, or macrophage), include but are not limited to use of a gapmer, shRNA, siRNA, a customized TALEN, meganuclease, or zinc finger nuclease, a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736; WO/2006/097853; WO/2003/087341;WO/2000/0415661; WO/2003/080809; WO/2010/079430; WO/2010/079430; WO/2011/072246;WO/2018/057989; and WO/2017/164230, the entire content of each of which are incorporated by reference.

Advantageously, guide RNA molecules comprising at least one guide sequence portion presented herein provide improved SARM1 knockout efficiency when complexed with a CRISPR nuclease in a cell relative to other guide RNA molecules. These specifically designed sequences may also be useful for identifying SARM1 target sites for other nucleotide targeting-based gene-editing or gene-silencing methods, for example, siRNA, TALENs, meganucleases or zinc-finger nucleases.

### Delivery to Cells

Any one of the compositions described herein useful for reducing or eliminating the expression of SARM1 may be delivered to a target cell by any suitable means. RNA guide molecules used to target SARM1 in methods of the present invention may be delivered to any cell intended to be administered to a subject for the purposes of cell therapy, or give rise to such a cell, and which contains and/or expresses a SARM1 allele. For example, in one embodiment of the present invention, an RNA molecule that specifically targets SARM1 alleles is delivered to a target cell and the target cell is a hematopoietic stem cell (HSC), induced pluripotent stem cells (iPS cells), natural killer cell (NK), iPS-derived NK cell (iNK), T cell, innate-like T cell (iT), natural killer T cell (NKT), invariant NKT cell (iNKT), monocyte, or macrophage. The delivery to the cell may be performed in vivo, ex vivo, or in vitro. In some embodiments, the delivery to the cell is ex vivo or in vitro, such that the generated modified cell may be administered to a subject in need thereof. In some embodiments, the delivery to the cell is in vivo, such that modification of the cell occurs in the subject in need thereof. Further, the nucleic acid compositions described herein may be delivered to a cell as one or more of DNA molecules, RNA molecules, ribonucleoproteins (RNP), nucleic acid vectors, or any combination thereof.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.™, Lipofectin.™ and Lipofectamine.™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow; and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

The disclosed compositions and methods may also be used in the manufacture of a medicament or immunotherapy for treating a cancer, disease, disorder, or infection in a patient.

### Examples of RNA Guide Sequence Portions which Specifically Target Alleles of SARM1 Gene

Although a large number of guide sequences can be designed to target the SARM1 gene, the nucleotide sequences described in Table 1 and are identified by SEQ ID NOs: 1-13457 were specifically selected to effectively implement the methods set forth herein. Examples of SARM1 biallelic knockout cells are provided in PCT International Application PCT/US2021/034583, the entire content of which is incorporated by reference. According to some aspects of the invention, the guide molecules disclosed herein may be utilized to generate SARM1 biallelic knockout cells exhibiting increased viability and/or increased functionality compared to counterpart cells that have wild-type or unmodified SARM1 expression. Non-limiting examples of cell types include liver cells (e.g., hepatocytes), lung cells, spleen cells, pancreas cells, colon cells, skin cells, bladder cells, eye cells, ocular cells, retinal cells, corneal cells, brain cells, esophagus cells, cells of the head, cells of the neck, cells of the ovary, cells of the testes, prostate cells, placenta cells, epithelial cells, endothelial cells, adipocyte cells, kidney/renal cells, heart cells, muscle cells, blood cells (e.g., white blood cells), immune cells, central nervous system (CNS) cells, ganglion cells, and the like, as well as combinations of the foregoing.

Table 1 shows guide sequences designed for use as described in the embodiments above to associate with SARM1 alleles. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SPRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequence portions designed to associate with specific SARM1 gene targets

| | SEQ ID NOs: of | SEQ ID NOs: of | SEQ ID NOs: of |
|---|---|---|---|
| | 20-nucleotide | 21-nucleotide | 22-nucleotide |
| | guide sequence | guide sequence | guide sequence |
| Target | portions | portions | portions |
| 17:28372165_G_C | 1-56 | 57-114 | 115-174 |
| rs782593684_REF | | | |
| 17:28372165_G_C | 1, 4, 8, 20, 24, | 57, 60, 64, 76, | 115, 118, 122, |
| rs782593684_SNP | 26-27, 33, 35, | 80, 82-83, 92, | 132, 139, 141- |
| | 37, 41, 47, 49, | 94, 98, 102, 105, | 142, 151, 157, |
| | 51, 53, 56, | 107, 109, 111, | 161, 164-165, |
| | 175-214 | 114, 215-256 | 167, 169, 171, |
| | | | 174, 257-300 |
| 17:28372349_C_CT | 301-356 | 357-414 | 415-474 |
| REF | | | |
| 17:28372349_C_CT | 301, 304, 315, | 357, 361, 364, | 415-416, 419, |
| SNP | 321, 326-328, | 368, 373, 384- | 422, 424, 427, |
| | 335, 338, 340, | 385, 393, 396, | 432, 443-444, |
| | 344, 346-348, | 398, 402, 404- | 458, 462, 464- |
| | 353, 355-356, | 406, 411, 413- | 466, 471, 473- |
| | 475-513 | 414, 514-554 | 474, 555-597 |
| 17:28372010-28372532 | 1-56, 301-356, | 57-114, 357-414, | 115-174, 415- |
| Exon 1, including the ATG start | 1348-2223 | 2224-3095 | 474, 3096-3963 |
| codon to 30 nt downstream of | | | |
| Exon 1 | | | |
| 17:28381173-28381851 | 3964-5203 | 5204-6443 | 6444-7683 |
| Exon 2, including 30 nt | | | |
| upstream and 30 nt downstream | | | |
| to Exon 2 | | | |
| 17:28384327-28384599 | 7684-8111 | 8112-8539 | 8540-8967 |
| Exon 3, including 30 nt | | | |
| upstream and 30 nt downstream | | | |
| to Exon 3 | | | |
| 17:28384809-28384960 | 8968-9153 | 9154-9339 | 9340-9525 |
| Exon 4, including 30 nt | | | |
| upstream and 30 nt downstream | | | |
| to Exon 4 | | | |
| 17:28385010-28385305 | 9526-9999 | 10000-10473 | 10474-10947 |
| Exon 5, including 30 nt | | | |
| upstream and 30 nt downstream | | | |
| to Exon 5 | | | |
| 17:28388144-28388306 | 10948-11155 | 11156-11363 | 11364-11571 |
| Exon 6, including 30 nt | | | |
| upstream and 30 nt downstream | | | |
| to Exon 6 | | | |
| 17:28388320-28388569 | 11572-11953 | 11954-12335 | 12336-12717 |
| Exon 7, including 30 nt | | | |
| upstream and 30 nt downstream | | | |
| to Exon 7 | | | |
| 17:28395875-28396056 | 12718-12963 | 12964-13209 | 13210-13455 |
| Exon 8, including 30 nt | | | |
| upstream and 30 nt downstream | | | |
| to Exon 8 | | | |
| 17:28396127-28396309 | 598-849 | 850-1099 | 1100-1347 |
| Exon 9, including 30 nt | | | |
| upstream of Exon 9 to the stop | | | |
| codon | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: SARM1 Knockout Anaylsis

Guide sequence portions comprising 17-50 contiguous nucleotides containing nucleotides in the sequence set forth in any one of SEQ ID NOs: 1-13457 are screened for high on target activity using SpCas9 in Hela cells. On target activity is determined by DNA capillary electrophoresis analysis.

### Example 2: Additional SARM1 Editing Anaylsis

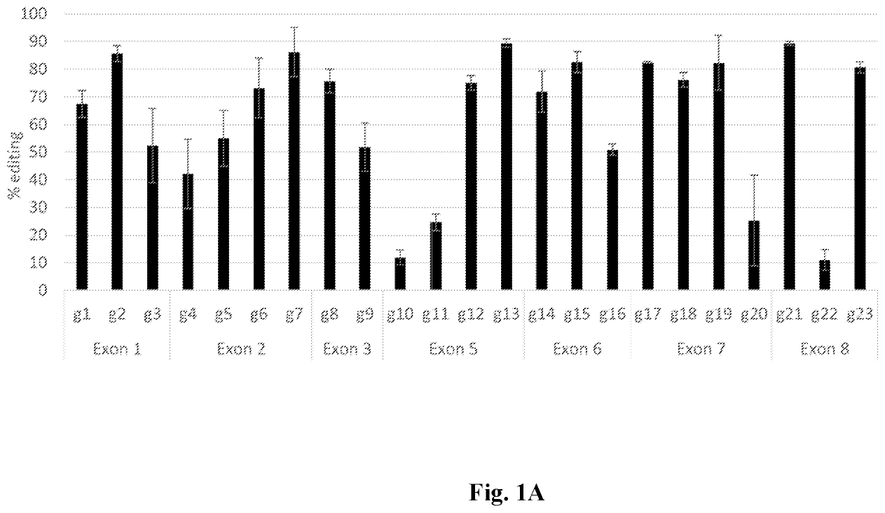

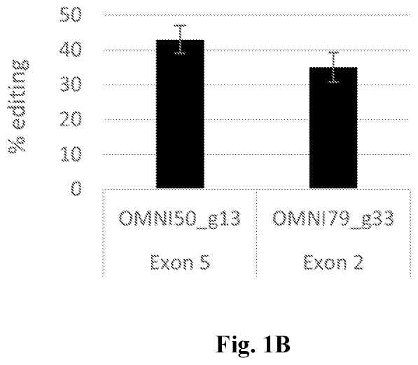

Sterile alpha and Toll/interleukin-1 receptor motif-containing 1 (SARM1) is NAD+ hydrolase whose activity is associated with axonal degeneration. To select optimal RNA guide molecules for biallelic knockout of SARM1, 23 RNA guide molecules targeting SARM1 exons were screened in Hela cells (Table 2). Briefly, an SpCas9 coding plasmid (64 ng) was co-transfected with a DNA plasmid that expresses a RNA guide molecule (20 ng) in a 96 well plate format using jetOPTIMUS® reagent (Polyplus). Cells were harvested 72 h post DNA transfection, genomic DNA was extracted and used for capillary electrophoresis using primers which amplify the endogenous genomic regions. The graphs in FIG. 1A represent the average of % editing±standard deviation (STDV) of three (3) independent experiments. Analysis of capillary electrophoresis data for all RNA guide molecules shows the activity ranges from 10% to 90%.

In addition, screens were performed with OMNI-50 (SEQ ID NO: 13471) and OMNI-79 (SEQ ID NO: 13472) CRISPR nucleases in Hela cells. Transfection conditions were identical to the conditions described for SpCas9 transfections. Editing efficiency was measured by next-generation sequencing (NGS) analysis. For OMNI-50, g13 editing efficiency was 43% (STDV=3.94). For OMNI-79, g33 editing efficiency was 35% (STDV=4.2). See FIG. 1B. Guide sequence portions of the RNA guide molecules are listed in Table 4.

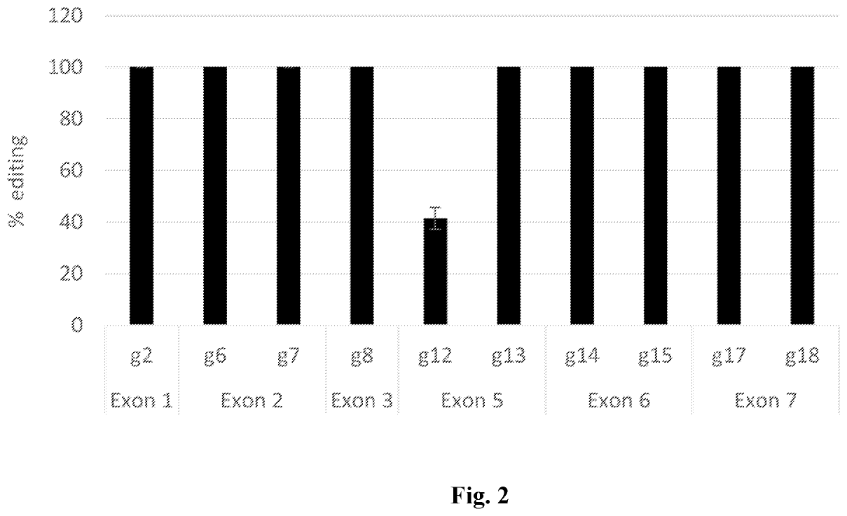

To validate RNA guide molecules conferring a Sarm1 knockout by non-sense mediated decay (NMD), mouse Neuro-2a cells which express SARM1 were used. To test the effect of the ten (10) most active guide RNA molecules that target human SARM1 from the HeLa screen, we identified ten (10) mouse specific guide RNA molecules that target mouse SARM1 DNA which correspond to the human guides RNA molecules. The activity of the mouse RNA guide molecules was tested in mouse cells. Briefly, 150×10³ Neuro-2a cells were mixed with pre-assembled RNPs composed of 105 pmole SpCas9 protein and 120 pmole sgRNAs (see Table 3), mixed with 100 pmole of electroporation enhancer (IDT-1075916), and electroporated using SF cell 4D-nucleofector X Kit S (PBC2-00675, Lonza) by applying the DS-134 program. A fraction of cells was harvested 72 hours post electroporation and genomic DNA was extracted to measure on-target activity by NGS. According to NGS analysis, all guide RNA molecules depicted high insertion or deletion (indel) activity (FIG. 2).

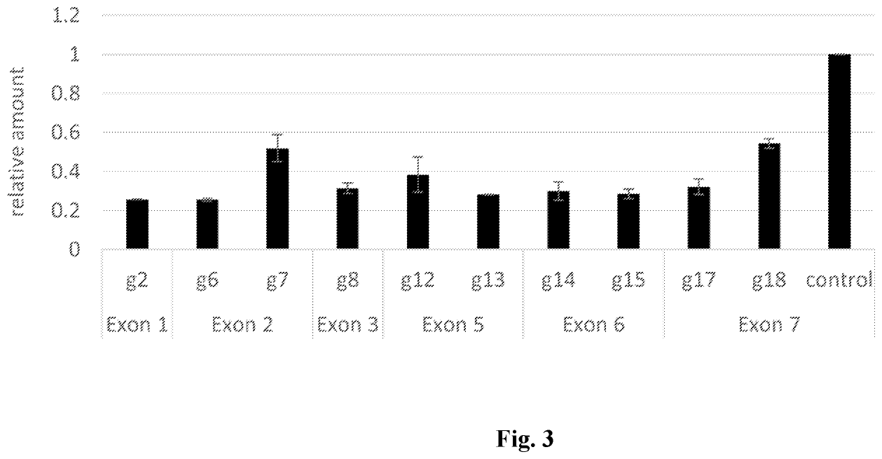

To assess the effect of the editing on the level of SARM1 transcripts, total RNA was extracted from Neuro-2a cells seven (7) days post electroporation and the mRNA level of Sarm1 was measured by qRT-PCR. The results demonstrate a more than an 80% reduction in the level of Sarm1 mRNA due to nonsense-mediated decay (NMD) (FIG. 3).

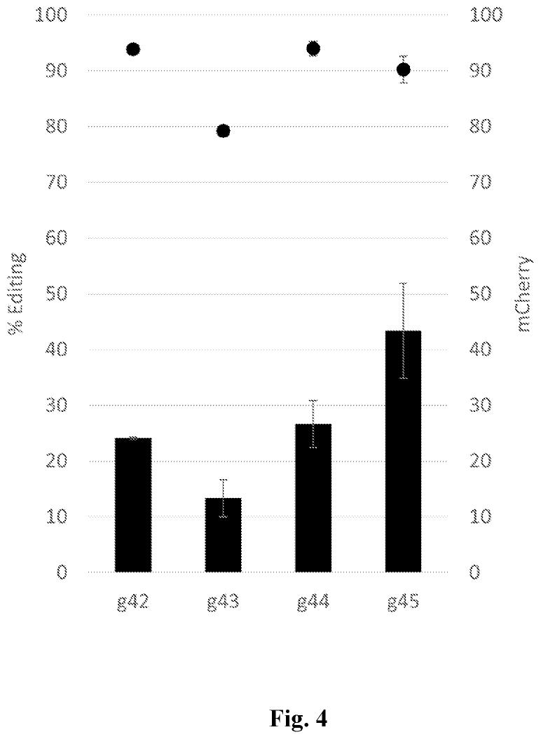

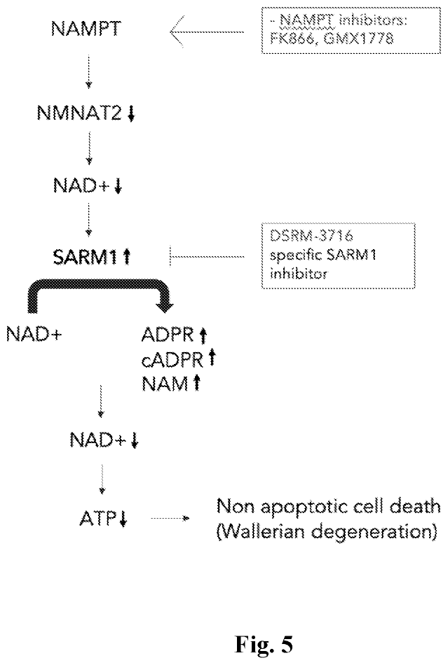

Next, a novel CRISPR nuclease, OMNI-103 (SEQ ID NO: 13473), which displays unique PAM requirements, was tested for editing of SARM1. This nuclease was tested in HeLa cells as described above. To this end, OMNI-103 was transfected into HeLa cells using a corresponding OMNI-P2A-mCherry expression vector (pmOMNI, Table 7) together with an sgRNA molecule designed to target a specific location in the human genome (guide sequence portion (gRNA) sequence listed in Table 5A). At 72 hours, cells were harvested, and half of the cells were used for quantification of transfection efficiency by FACS using mCherry fluorescence as a marker. The rest of the cells were lysed, and their genomic DNA content was used in a PCR reaction which amplified the corresponding putative genomic targets. Amplicons were subjected to NGS and the resulting sequences were then used calculate the percentage of editing events in each target site. Short Insertions or deletions (indels) around the cut site are the typical outcome of repair of DNA ends following nuclease induced DNA cleavage. The calculation of % editing was therefore deduced from the fraction of indels containing sequences within each amplicon. See Table 5B and FIG. 4.

### Example 3: SARM1-Edited NK Cell Assays

### Metabolic Assays

Glycolysis and oxidative mitochondrial metabolism: Seahorse assays will be performed according to the manufacturer's instructions with modifications to simultaneously analyze glycolysis and oxidative mitochondrial metabolism using the Seahorse XF Glycolysis Stress Test Kit and the Seahorse XF Cell Mito Stress Kit (Agilent Technologies). Briefly, NK and iNK cells are washed and resuspended in glucose-free media (GIBCO). 1.5×10⁵ cells are plated per well in triplicate and analyzed with a Seahorse Xfe96 Analyzer (Agilent Technologies). Glucose, oligomycin, FCCP, sodium pyruvate, rotenone, and antimycin A are serially injected to measure metabolic function. SRC measurements are calculated as average maximal OCR values minus average basal OCR values. ATP-linked respiration is calculated as average basal OCR values minus average post-oligomycin values. Glycolysis is calculated as average post-glucose ECAR values minus average basal ECAR values. Glycolytic reserve is calculated as average maximal ECAR values minus post-glucose ECAR values.

ATP quantification assays: 1×10⁵ NK and iNK cells per well are analyzed using the ATP Bioluminescence Assay Kit HS II (Sigma Aldrich) and analyzed with an Infinite M200 PRO Luminometer (Tecan).

NAD+ and NADH quantification assays: NAD+ and NADH concentrations are quantified using the NAD/NADH Cell-Based Assay Kit (Cayman Chemical) as per the manufacturer's instructions and analyzed with an Infinite M200 PRO Luminometer. For analyses of oxidative stress, NK and iNK cells are cultured with hydrogen peroxide (Sigma Aldrich) for 1 hour. Following treatment, cells are cultured in serum-free media containing 5 mM MitoSox Indicator Dye (Thermo Fisher). Cells are then washed and counter stained with anti-CD56 antibody and fixable viability dye for flow cytometry analysis. For mass spectrometry analysis of metabolites, iNK cells and expanded peripheral blood NK cells are snap frozen in liquid nitrogen and metabolomics is performed.

### Killing Assays

In vitro assays to assess cytotoxicity: Tumor target cells are CFSE-stained and resuspended in media containing 5% FBS. NK effector cells are co-cultured with the target cells in 96-well plate at 5:1 E:T ratio and in the presence of Propidium Iodide. The cocultured cells are placed in the IncuCyte (Sartorius), live cell analysis system, for up to 24 hours and the killing ability of the NK cells are quantified by of the double-stained cells, which indicates dead target cells.

3-dimensional tumor spheroid cytotoxicity assays: Tumor cells are cultured for a few day's to allow for spheroid formation. Next, 4×10⁴ NK or iNK cells were gently added to each well and cells were co-cultured for few days. At the end of the culture, cells in each well are disrupted into a single cell suspension and stained with a fluorescently conjugated CD56 antibody and fixable viability dye for flow cytometry analysis. Tumor cells are quantified based on NucLight Red, and NK or iNK cells are quantified based on CD56 expression.

### In Vivo Retention Model of SARM1-Edited NK Cells:

In vivo functionality of the edited NK cells will be tested using retention model in NSG mice. Three experimental groups (control, edited cells and unedited cells) are used. The mice will be irradiated with 300 rad a day before the injection with the cells. The cells will be resuspended in 200 μl buffer and injected to the mice intravenously. IL2 and IL15 will be administrated via intraperitoneal injection. The mice will be sacrificed 3 to 4 days post-injection, and bone-marrow and spleen will be analyzed to measure the NK retention ability. A table summarizing the assay is shown below:

| | | | | Cytokines dose | | |
|---|---|---|---|---|---|---|
| | | NK cells | | (μg/200 μl/mouse) | | No. of |
| Grp | Description | Cell dose | Inj. Vol (μl) | IL2 | IL15 | mice |
| A | HSA buffer (control) | — | 200 μl | 5 | 0.5 | 4 |
| B | Unedited cells | 10 × 10⁶ | | | | 10 |
| C | Edited cells | 10 × 10⁶ | | | | 10 |

TABLE 2
20-nucleotide guide sequence portion sequences targeting human
SARM1 coding sequence

| Guide | | |
|---|---|---|
| sequence | | |
| portion | | |
| (gRNA) | Sequence of gRNA | PAM |
| g1 | GGCCCAUGGUGGGCUGCGGG (SEQ ID NO: 39) | UGG |
| g2 | GCGCCUGCUGGAGCAGAUCC (SEQ ID NO: 1927) | UGG |
| g3 | CUCCACCAGUUGGAAGACCU (SEQ ID NO: 1745) | CGG |
| g4 | AGAGACCGCGUGGCGCGCAU (SEQ ID NO: 13456) | UGG |
| g5 | CGUGAUCCUGAACCUGGCGA (SEQ ID NO: 4489) | AGG |
| g6 | GGCAACUGCGCGCUGCACGG (SEQ ID NO: 4870) | GGG |
| g7 | GGCGAGCGGGAAGAGCCACU (SEQ ID NO: 4897) | CGG |
| g8 | CAUCCAGAGCCUGAAACGCC (SEQ ID NO: 7785) | UGG |
| g9 | UGCUGGGCGAGGAGGUGCCA (SEQ ID NO: 8084) | CGG |
| g10 | AGCAACCUGGCGGACUGGCU (SEQ ID NO: 9562) | GGG |
| g11 | GGCGGACUGGCUGGGCAGCC (SEQ ID NO: 9868) | UGG |
| g12 | ACACGCGGUGCAGCAGGGAG (SEQ ID NO: 9534) | CGG |
| g13 | CUCAGACACGCGGUGCAGCA (SEQ ID NO: 9726) | GGG |
| g14 | CACUCCCCGCUGCCCUGUAC (SEQ ID NO: 10989) | UGG |
| g15 | UCCCCGCUGCCCUGUACUGG (SEQ ID NO: 11127) | UGG |
| g16 | UGCCACCAGUACAGGGCAGC (SEQ ID NO: 11136) | GGG |
| g17 | AAGGUGCACCUGCAGCUGCA (SEQ ID NO: 11586) | UGG |
| g18 | CAUGCAAGACCAUGACUGCA (SEQ ID NO: 11704) | AGG |
| g19 | GCAGCUGCAGGUGCACCUUC (SEQ ID NO: 13457) | AGG |
| g20 | GCACCCAAUCCUUGCAGUCA (SEQ ID NO: 11795) | UGG |
| g21 | AUUGUGACUGCUUUAAGCUG (SEQ ID NO: 12768) | CGG |
| g22 | AACAUUGUGCCCAUCAUUGA (SEQ ID NO: 12719) | UGG |
| g23 | CACUCGAAGCCAUCAAUGAU (SEQ ID NO: 12777) | GGG |

TABLE 3
20-nucleotide guide sequence portion sequences targeting mouse
Sarm1 coding sequence

| Guide | | |
|---|---|---|
| sequence | | |
| portion | | |
| (gRNA) | Sequence of gRNA | PAM |
| g2 mouse | GCGCUUGCUGGAGCAGAUCC (SEQ ID NO: 13463) | UGG |
| g6 mouse | GCGAACUGCGCGCUGCACGG (SEQ ID NO: 13464) | GGG |
| g7 mouse | AGCGAGCGGGAAGAGCCACU (SEQ ID NO: 13465) | CGG |
| g8 mouse | UAUCCAGAGCCUGAAACGCC (SEQ ID NO: 13466) | UGG |
| g12 mouse | ACACGCGGUGCAGCAGGGAG (SEQ ID NO: 9534) | CGG |
| g13 mouse | CUCUGACACGCGGUGCAGCA (SEQ ID NO: 13467) | GGG |
| g14 mouse | CAUUCCCCGCUGCCCUGUAC (SEQ ID NO: 13468) | UGG |
| g15 mouse | UCCCCGCUGCCCUGUACUGG (SEQ ID NO: 11127) | AGG |
| g17 mouse | AAGGUGCACCUGCAGCUUCA (SEQ ID NO: 13469) | CGG |
| g18 mouse | CAUGCAGGACCAUGACUGCA (SEQ ID NO: 13470) | AGG |

TABLE 4
22-nucleotide guide sequence portion sequences targeting human
SARM1 coding sequence

| Guide | | | |
|---|---|---|---|
| sequence | | | |
| portion | | | |
| (gRNA) | Sequence of gRNA | PAM | CRISPR nuclease |
| g13 | UGCUCAGACACGCGGUGCAGCA | GGG | OMNI-50 |
| | (SEQ ID NO: 9803) | | |
| g33 | GUAGCGGUGUUGGCGACUAACA | AGG | OMNI-79 |
| | (SEQ ID NO: 6567) | | |

TABLE 5A
22-nucletoide guide sequence portion sequences targeting sequences
located in SARM1 region

| Guide sequence | |
|---|---|
| portion (gRNA) | Sequence of guide sequence portion |
| g42 | CGCGCGGCCUGCACACGCGUCU (SEQ ID NO: 3422) |
| g43 | CGCCACUGCGCGCUGGCGCUGG (SEQ ID NO: 6877) |
| g44 | GUGUCUGAGCAGCAGCUGCUGG (SEQ ID NO: 10869) |
| g45 | GAUGUCUUCAUCAGCUACCGCC (SEQ ID NO: 11485) |

TABLE 5B
Quantitative results depicted in FIG. 4

| | % Editing | % mCherry | STDV ″% Editing″ | STDV ″% mCherry″ |
|---|---|---|---|---|
| g42 | 24.1566667 | 93.8333333 | 0.215019379 | 0.450924975 |
| g43 | 13.3566667 | 79.2333333 | 3.303957223 | 0.550757055 |
| g44 | 26.6933333 | 94 | 4.196431023 | 1.322875656 |
| g45 | 43.415 | 90.2 | 8.548920985 | 2.4 |

TABLE 6
Novel OMNI CRISPR nucleases, PAM requirements, and sgRNA
scaffold sequences

| OMNI CRISPR | PAM | |
|---|---|---|
| Nuclease and gRNA | Sequence | sgRNA Scaffold Sequence |
| OMNI-50 | NGG | UGCUCUGACACGCGGUGCAGCAGUUUGAGAGUU |
| (SEQ ID NO: 13471) | | AUGUAAGAAAUUACAUGACGAGUUCAAAUAAAA |
| with g13 | | AUUUAUUCAAACCGCCUAUUUAUAGGCCGCAGA |
| | | UGUUCUGCAUUAUGCUUGCUAUUGCAAGCUUUU |
| | | UU (SEQ ID NO: 13474) |
| OMNI-79 | NGR | GUAGCGGUGUUGGCGACUAACAGUUGCCGCUGG |
| (SEQ ID NO: 13472) | | AGAAAUCCAGUUGUUAACAAGCAGCUUGACUGC |
| with g33 | | ACCAAAUAAGGCGGGGGCUGCGGCCCUCGCUUU |
| | | UUU (SEQ ID NO: 13475) |
| OMNI-103 | NNRACT | CGCCACUGCGCGCUGGCGCUGGGUUUGAGAGUA |
| (SEQ ID NO: 13473) | | GUGUAAGAAAUUACACUACAAGUUCAAAUAAAA |
| with g42 | | AUUUAUUCAAAUCCAUUUGCUACAUUGUGUAGA |
| | | AUUUAAAGAUCUGGCAACAGAUCUUUUUUU (SEQ |
| | | ID NO: 13476) |
| OMNI-103 | NNRACT | GUGUCUGAGCAGCAGCUGCUGGGUUUGAGAGUA |
| (SEQ ID NO: 13473) | | GUGUAAGAAAUUACACUACAAGUUCAAAUAAAA |
| with g43 | | AUUUAUUCAAAUCCAUUUGCUACAUUGUGUAGA |
| | | AUUUAAAGAUCUGGCAACAGAUCUUUUUUU (SEQ |
| | | ID NO: 13477) |
| OMNI-103 | NNRACT | GAUGUCUUCAUCAGCUACCGCCGUUUGAGAGUA |
| (SEQ ID NO: 13473) | | GUGUAAGAAAUUACACUACAAGUUCAAAUAAAA |
| with g44 | | AUUUAUUCAAAUCCAUUUGCUACAUUGUGUAGA |
| | | AUUUAAAGAUCUGGCAACAGAUCUUUUUUU (SEQ |
| | | ID NO: 13478) |
| OMNI-103 | NNRACT | UGCUCUGACACGCGGUGCAGCAGUUUGAGAGUU |
| (SEQ ID NO: 13473) | | AUGUAAGAAAUUACAUGACGAGUUCAAAUAAAA |
| with g45 | | AUUUAUUCAAACCGCCUAUUUAUAGGCCGCAGA |
| | | UGUUCUGCAUUAUGCUUGCUAUUGCAAGCUUUU |
| | | UU (SEQ ID NO: 13479) |

TABLE 7
OMNI CRISPR nuclease mammalian expression plasmid and elements

| Plasmid | | |
|---|---|---|
| Name | Purpose | Elements |
| pmOMNI | Expressing OMNI polypeptide | CMV promoter-Kozak-SV40 NLS- |
| | in the mammalian system | OMNI ORF (human optimized)-HA-SV40 |
| | | NLS-P2A-mCherry-bGH poly(A) signal |

TABLE 7
Annex

| | SEQ ID NO of Amino | SEQ ID NO of DNA |
|---|---|---|
| Element | Acid Sequence | sequence |
| HA Tag | SEQ ID NO: 13480 | SEQ ID NO: 13484 |
| NLS | SEQ ID NO: 13481 | SEQ ID NO: 13485 |
| P2A | SEQ ID NO: 13482 | SEQ ID NO: 13486 |
| mCherry | SEQ ID NO: 13483 | SEQ ID NO: 13487 |

### Example 4: Knockout of SARM1 in NK Cells Improves Killing Activity of the NK Cells

### Introduction

SARM1, Sterile alpha and Toll/interleukin-1 receptor motif-containing 1, is NAD+ hydrolase whose activity is associated with axonal degeneration. GMX1778 is a potent and specific inhibitor of the NAD+ biosynthesis enzyme nicotinamide phosphoribosyltransferase (NAMPT). Selective inhibition of NAMPT by GMX1778 blocks the production of NAD+ and results in cell death. Thus, NAMPT represents an important controller of intracellular NAD concentrations, and consequently, also of energy metabolism. See FIG. 5.

### Results

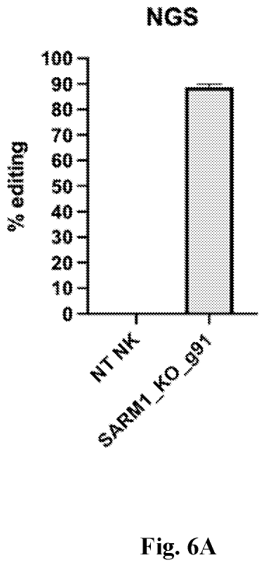

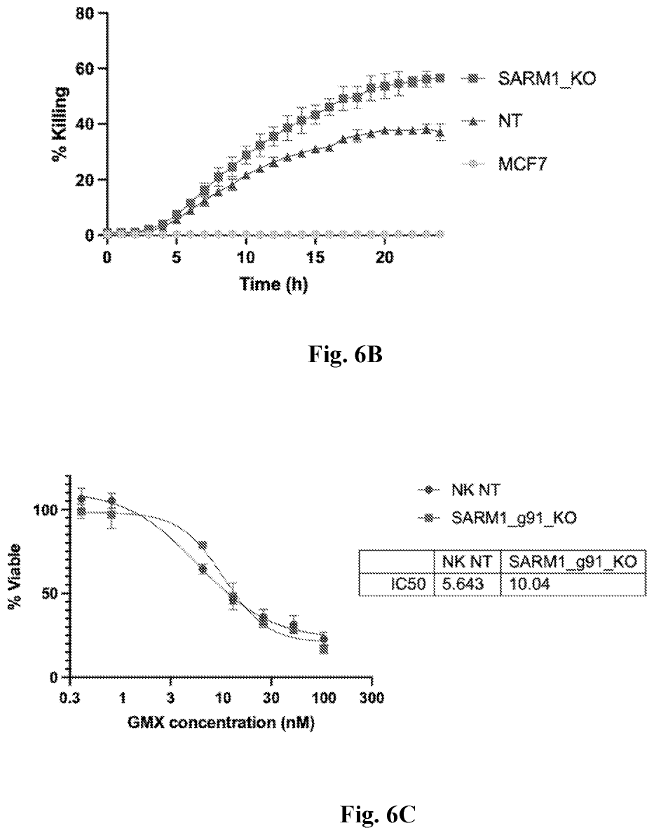

Editing and NGS: 2×10⁶ thawed primary NK cells were grown for five (5) days and were then electroporated with 113 pmol SpCas9 and 226 pmol SARM1 g91 gRNA (GUACUGGUGGCAAACCCAGU (SEQ ID NO: 11104)). After seven (7) days, cells were sent for NGS analysis, which showed 90% editing of SARM1. See FIG. 6A.

Killing assay: Ten (10) days after electroporation, 6250 NK cells were plated with 2,500 MCF7 target cells labeled with GFP, in triplicate. Co-cultures were incubated for 24 hours in 2.5:1 E:T ratio. Images were taken every hour by Incucyte®. Killing assay medium was MEM-alpha supplemented with 10% FBS and 20 ng/ml IL-15. See FIG. 6B.

Viability (ATPlite) assay: Fifteen (15) days after electroporation, 50,000 SARM1_g91_KO and non-treated (NT) cells were plated in triplicate with increasing concentrations of GMX1778 and were incubated for 48 hours. Cell viability was quantified by the ATPlite kit. See FIG. 6C.

### Conclusions

These results indicate that SARM1-knockout NK cells show improvement in cell viability upon GMX1778 treatment (SARM1 inducer). The results further demonstrate that SARM1-knockout NK cells show improvement in killing ability.

Thus, the results of this Examples represent a proof-of-concept that knockout of SARM1 in NK cells improves killing activity of the NK cells. Other nucleases and guide molecule combinations may be used to knockout SARM1 and achieve the same effect. These unexpected results are the first showing that knocking out SARM1 in NK cells improves the NK cells' killing activity. Thus, SARM1-KO NK cells may be used in immunotherapies, for example, cancer treatments.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
4. Behr (1994) Gene transfer with synthetic cationic amphiphiles: Prospects for gene therapy”, Bioconjuage Chem 5:382-89.
5. Blaese (1995) “Vectors in cancer therapy: how will they deliver”, Cancer Gene Ther. 2:291-97.
6. Blaese et al. (1995) “T lympocyte-directed gene therapy for ADA-SCID: initial trial results after 4 years”, Science 270(5235):475-80.
7. Buchschacher and Panganiban (1992) “Human immunodeficiency virus vectors for inducible expression of foreign genes”, J. Virol. 66:2731-39.
8. Burstein et al. (2017) “New CRISPR-Cas systems from uncultivated microbes”, Nature 542:237-41.
9. Chang and Wilson (1987) “Modification of DNA ends can decrease end-joining relative to homologous recombination in mammalian cells”, Proc. Natl. Acad. Sci. USA 84:4959-4963.
10. Chung et al. (2006) “Agrobacterium is not alone: gene transfer to plants by viruses and other bacteria”, Trends Plant Sci. 11(1):1-4.
11. Coelho et al. (2013) “Safety and efficacy of RNAi therapy for transthyretin amyloidosis” N. Engl. J. Med. 369, 819-829.
12. Collias and Beisel (2021) “CRISPR technologies and the search for the PAM-free nuclease”, Nature Communications 12, 555.
13. Crystal (1995) “Transfer of genes to humans: early lessons and obstacles to success”, Science 270(5235):404-10.
14. Dillon (1993) “Regulation gene expression in gene therapy” Trends in Biotechnology 11(5):167-173.
15. Dranoff et al. (1997) “A phase I study of vaccination with autologous, irradiated melanoma cells engineered to secrete human granulocyte macrophage colony stimulating factor”, Hum. Gene Ther. 8(1):111-23.
16. Dunbar et al. (1995) “Retrovirally marked CD34-enriched peripheral blood and bone marrow cells contribute to long-term engraftment after autologous transplantation”, Blood 85:3048-57.
17. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
18. Gao and Huang (1995) “Cationic liposome-mediated gene transfer” Gene Ther. 2(10):710-22.
19. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
20. Han et al. (1995) “Ligand-directed retro-viral targeting of human breast cancer cells”, Proc Natl Acad Sci U.S.A. 92(21):9747-51.
21. Hu et al. (2018) “Evolved Cas9 variants with broad PAM compatibility and high DNA specificity”, Nature 556(7699):57-63.
22. Jinek et al. (2012) “A programmable dual-RNA-guided DNA endonuclease in adaptive bacterial immunity,” Science 337(6096):816-21.
23. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
24. Judge et al. (2006) “Design of noninflammatory synthetic siRNA mediating potent gene silencing in vivo”, Mol Ther. 13(3):494-505.
25. Kohn et al. (1995) “Engraftment of gene-modified umbilical cord blood cells in neonates with adnosine deaminase deficiency”, Nature Medicine 1:1017-23.
26. Kremer and Perricaudet (1995) “Adenovirus and adeno-associated virus mediated gene transfer”, Br. Med. Bull. 51(1):31-44.
27. Macdiarmid et al. (2009) “Sequential treatment of drug-resistant tumors with targeted minicells containing siRNA or a cytotoxic drug”, Nat Biotehcnol. 27(7):643-51.
28. Malech et al. (1997) “Prolonged production of NADPH oxidase-corrected granulocyes after gene therapy of chronic granulomatous disease”, PNAS 94(22):12133-38.
29. Miller et al. (1991) “Construction and properties of retrovirus packaging cells based on gibbon ape leukemia virus”, J Virol. 65(5):2220-24.
30. Miller (1992) “Human gene therapy comes of age”, Nature 357:455-60.
31. Mitani and Caskey (1993) “Delivering therapeutic genes—matching approach and application”, Trends in Biotechnology 11(5):162-66.
32. Molday et al. (2013) “X-linked juvenile retinoschisis: Clinical diagnosis, genetic analysis, and molecular mechansims”, Prog Retin Eye Res. 31(3):195-212.
33. Nabel and Felgner (1993) “Direct gene transfer for immunotherapy and immunization”, Trends in Biotechnology 11(5):211-15.
34. Nishimasu et al. (2018) “Engineered CRISPR-Cas9 nuclease with expanded targeting space”, Science 361(6408):1259-1262.
35. Remy et al. (1994) “Gene Transfer with a Series of Lipphilic DNA-Binding Molecules”, Bioconjugate Chem. 5(6):647-54.
36. Sentmanat et al. (2018) “A Survey of Validation Strategies for CRISPR-Cas9 Editing”, Scientific Reports 8:888, doi:10.1038/s41598-018-19441-8.
37. Sommerfelt et al. (1990) “Localization of the receptor gene for type D simian retroviruses on human chromosome 19”, J. Virol. 64(12):6214-20.
38. Van Brunt (1988) “Molecular framing: transgenic animals as bioactors” Biotechnology 6:1149-54.
39. Vigne et al. (1995) “Third-generation adenovectors for gene therapy”, Restorative Neurology and Neuroscience 8(1,2):35-36.
40. Walton et al. (2020) “Unconstrained genome targeting with near-PAMless engineered CRISPR-Cas9 variants”, Science 368(6488):290-296.
41. Weissman and Kariko (2015) “mRNA: Fulfilling the promise of gene therapy”, Molecular Therapy (9):1416-7.
42. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
43. Yu et al. (1994) “Progress towards gene therapy for HIV infection”, Gene Ther. 1(1):13-26.
44. Zetsche et al. (2015) “Cpf1 is a single RNA-guided endonuclease of a class 2 CRIPSR-Cas system” Cell 163(3):759-71.
45. Zuris et al. (2015) “Cationic lipid-mediated delivery of proteins enables efficient protein based genome editing in vitro and in vivo” Nat Biotechnol. 33(1):73-80.

1. A method for adoptive cell therapy or prophylaxis comprising administering SARM1-inhibited or SARM1-inactivated cells to a subject suffering from or determined to be at risk of suffering from a cancer, infection, disease, or disorder.

2. The method of claim 1, wherein the SARM1-inhibited or SARM1-inactivated cells are modified to have reduced or inactivated expression of SARM1, or the cells are modified to express a dominant negative SARM1 sequence variant or dominant negative fragment thereof.

3. The method of claim 1 or 2, wherein the cells are selected from the group consisting of hematopoietic stem cells (HSCs), induced pluripotent stem cells (iPS cells), iPSc-derived cells, natural killer cells (NKs), iPS-derived NK cells (iNKs), T cells, innate-like T cells (iTs), natural killer T cells (NKTs), γδ T cells, iPSc-derived T cells, invariant NKT cells (iNKTs), iPSc-derived NKTs, monocytes, or macrophages.

4. The method of any one of claims 1-3, wherein the SARM1-inhibited or SARM1-inactivated cells display increased functionality, increased viability, increased persistence, increased proliferation, and/or increased tumor retention in the subject relative to counterpart cells that have wild-type or unmodified SARM1 expression.

5. The method of any one of claims 1-4, wherein the SARM1-inhibited or SARM1-inactivated cells display increased cytotoxic activity and/or increased killing activity in the subject relative to counterpart cells that have wild-type or unmodified SARM1 expression.

6. The method of any one of claims 1-5, wherein the subject is suffering from or determined to be at risk of suffering from a cancer.

7. The method of claim 6, wherein the cancer comprises a tumor and/or the cancer is a hematological malignancy.

8. The method of claim 7, wherein the cancer is selected from the group consisting of static melanoma, metastatic prostate cancer, metastatic breast cancer, triple negative breast cancer, bladder cancer, brain cancer, esophageal cancer, liver cancer, head and neck cancer, squamous cell lung cancer, non-small lung cell cancer, Merkel cell carcinoma, sarcoma, hepatocellular cancer, multiple myeloma, leukemia, non-Hodgkin's lymphoma, lymphoma, B cell lymphoma, acute myeloid leukemia, pancreatic cancer, colorectal carcinoma, cervical cancer, gastric carcinoma, kidney cancer, metastatic renal cell carcinoma, leukemia, ovarian cancer, and malignant glioma.

9. The method of any one of claims 1-8, wherein the SARM1-inhibited or SARM1-inactivated cells are created ex vivo or in vitro.

10. The method of any one of claims 1-8, wherein the SARM1-inhibited or SARM1-inactivated cells are created in vivo.

11. The method of any one of claims 1-10, wherein the SARM1-inhibited or SARM1-inactivated cells are created from cells obtained from the subject by mobilization and/or by apheresis.

12. The method of claim 11, wherein the cells are obtained the cells from the subject by bone marrow aspiration.

13. The method of any one of claims 1-12, wherein the cells are prestimulated prior to the SARM1 inhibition or SARM1 inactivation of the cells.

14. The method of any one of claims 1-13, wherein the SARM1-inhibited or SARM1-inactivated cells are culture expanded prior to administration to the subject.

15. The method of any one of claims 1-14, wherein the SARM1-inhibited or SARM1-inactivated cells are capable of engraftment.

16. The method of any one of claims 1-15, wherein the SARM1-inhibited or SARM1-inactivated cells are capable of giving rise to progeny cells.

17. The method of claim 16, wherein the SARM1-inhibited or SARM1-inactivated cells are capable of giving rise to progeny cells after an engraftment.

18. The method of claim 17, wherein the SARM1-inhibited or SARM1-inactivated cells are capable of giving rise to progeny cells after an autologous engraftment.

19. The method of claim 17 or 18, wherein the SARM1-inhibited or SARM1-inactivated cells are capable of giving rise to progeny cells for at least 12 months or at least 24 months after engraftment.

20. The method of any one of claims 1-19, wherein the SARM1-inhibited or SARM1-inactivated cells are created by delivering a gapmer, shRNA, siRNA, a customized TALEN, meganuclease, zinc finger nuclease, CRISPR nuclease, or a small molecule inhibitor to cells.

21. The method of any one of claims 1-19, wherein alleles of the SARM1 gene in the SARM1-inhibited or SARM1-inactivated cells are subjected to an insertion or deletion mutation.

22. The method of claim 21, wherein the insertion or deletion mutation creates an early stop codon.

23. The method of any one of claims 1-22, wherein SARM1-inhibited or SARM1-inactivated cells are created by a method comprising
introducing to the cells a composition comprising:
at least one CRISPR nuclease, or a nucleotide molecule encoding a CRISPR nuclease; and
an RNA molecule comprising a guide sequence portion, or a nucleotide molecule encoding the RNA molecule,
wherein a complex of the CRISPR nuclease and the RNA molecule affects a double strand break in alleles of the SARM1 gene,
wherein the guide sequence portion of the RNA molecule comprises 17-50 contiguous nucleotides.

24. The method of claim 23, wherein guide sequence portion is complementary to a target sequence located from 50 base pairs upstream to 50 base pairs downstream of Exon I, Exon II, Exon III, Exon IV, Exon V, Exon VI, Exon VII, Exon VIII, or Exon IX of the SARM1 gene.

25. The method of claim 23 or 24, wherein the guide sequence portion is complementary to a target sequence located from 30 base pairs upstream to 30 base pairs downstream of an Exon of the SARM1 gene, and
a) the Exon is Exon I and the guide sequence portion comprises a sequence that is the same as or differs by no more than 3 nucleotides from a sequence set forth in any of SEQ ID NOs: 1-56, 301-356, 1348-2223, 57-114, 357-414, 2224-3095, 115-174, 415-474, 3096-3963;
b) the Exon is Exon II, and the guide sequence portion comprises a sequence that is the same as or differs by no more than 3 nucleotides from a sequence set forth in any of SEQ ID NOs: 3964-7683;
c) the Exon is Exon III, and the guide sequence portion comprises a sequence that is the same as or differs by no more than 3 nucleotides from a sequence set forth in any of SEQ ID NOs: 7684-8967;
d) the Exon is Exon IV, and the guide sequence portion comprises a sequence that is the same as or differs by no more than 3 nucleotides from a sequence set forth in any of SEQ ID NOs: 8968-9525;
e) the Exon is Exon V, and the guide sequence portion comprises a sequence that is the same as or differs by no more than 3 nucleotides from a sequence set forth in any of SEQ ID NOs: 9526-10947;
f) the Exon is Exon VI, and the guide sequence portion comprises a sequence that is the same as or differs by no more than 3 nucleotides from a sequence set forth in any of SEQ ID NOs: 10948-11571;
g) the Exon is Exon VII, and the guide sequence portion comprises a sequence that is the same as or differs by no more than 3 nucleotides from a sequence set forth in any of SEQ ID NOs: 11572-12717;
h) the Exon is Exon VIII, and the guide sequence portion comprises a sequence that is the same as or differs by no more than 3 nucleotides from a sequence set forth in any of SEQ ID NOs: 12718-13455; or
i) the Exon is Exon IX, and the guide sequence portion comprises a sequence that is the same as or differs by no more than 3 nucleotides from a sequence set forth in any of SEQ ID NOs: 598-1347.

26. The method of any one of claims 23-25, wherein the guide sequence portion comprises 17-50 contiguous nucleotides containing nucleotides in the sequence set forth in any one of SEQ ID NOs: 1-13457.

27. A medicament comprising SARM1-inhibited or SARM1-inactivated cells for use in treating or preventing a cancer, infection, disease, or disorder in a subject according to the method of any one of claims 1-26.

28. A kit for treating or preventing a cancer, infection, disease, or disorder in a subject, comprising the medicament of claim 27 and instructions for delivering the composition to a subject suffering from or determined to be at risk of suffering from a cancer, infection, disease, or disorder.

29. A method for inactivating alleles of the sterile alpha and toll/interleukin-1 receptor motif-containing 1 (SARM1) gene in a cell, the method comprising
introducing to the cell a composition comprising:
at least one CRISPR nuclease, or a nucleotide molecule encoding a CRISPR nuclease; and
an RNA molecule comprising a guide sequence portion, or a nucleotide molecule encoding the RNA molecule,
wherein a complex of the CRISPR nuclease and the RNA molecule affects a double strand break in alleles of the SARM1 gene,
wherein the guide sequence portion comprises 17-50 contiguous nucleotides containing nucleotides in the sequence set forth in any one of SEQ ID NOs: 1-13457, and
wherein the cell is selected from the group consisting of a hematopoietic stem cell (HSC), induced pluripotent stem cell (iPS cell), iPSc-derived cell, natural killer cell (NK), iPS-derived NK cell (iNK), T cell, innate-like T cell (iT), natural killer T cell (NKT), γδ T cell, iPSc-derived T cell, invariant NKT cell (iNKT), iPSc-derived NKT, monocyte, or macrophage.

30. A cell modified by the method of claim 29, wherein the modified cell comprises at least one inactivated SARM1 allele.

31. The modified cell of claim 30 for use in adoptive cell therapy or prophylaxis.

32. The modified cell of claim 31, wherein the adoptive cell therapy or prophylaxis is to treat or prevent a cancer, infection, disease, or disorder in a subject.

33. A composition, method, process, kit, modified cell, or use as characterized by one or more elements disclosed herein.
